(19) United States
(12) Patent Application Publication
(10) Pub. No.: US 2008/0194608 A1
(43) Pub. Date: Aug. 14, 2008

(54) 6-Phenylhex-5-Enoic Acid Derivatives, Process for the Preparation Thereof, Pharmaceutical Compositions Comprising Them, and Therapeutic Uses Thereof

(76) Inventors: Michel Brunet, Toussieu (FR); Herve Dumas, Moules et Baucels (FR); Catherine Vidal, Lyon (FR); Nathalie Adje, Genas (FR); Benoit Van Hille, Collonges au Mont D'Or (FR); Francis Contard, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/813,927

(22) PCT Filed: Dec. 22, 2005
(86) PCT No.: PCT/EP2005/013857

§ 371 Date: Jul. 13, 2007

(30) Foreign Application Priority Data
Jan. 14, 2005 (FR) 0500419

(51) Int. Cl. A61K 31/44; C07C 69/00; A61K 31/216; C07C 59/00; A61K 31/19; C07D 263/02; A61P 9/10; A61P 3/10; A61K 31/421; C07D 333/50; A61K 31/381; C07D 213/02

(52) U.S. Cl. 514/277; 560/55; 514/532; 562/465; 514/568; 548/215; 514/374; 549/49; 514/443; 546/339

(57) ABSTRACT

Compounds of the formula (I): in which R¹, R², R³, R′³ and R⁴ are as defined in the description, the use thereof for the treatment of dyslipidaemia, atherosclerosis and diabetes, the pharmaceutical compositions comprising them, and the processes for the preparation of these compounds.

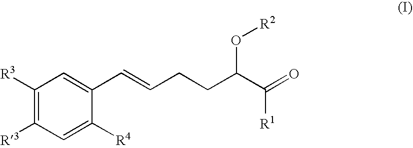

The present invention relates to 6-phenylhex-5-enoic acid derivatives that can be used in the treatment of dyslipidaemia, atherosclerosis and diabetes. The invention also relates to pharmaceutical compositions comprising them and to processes for the preparation of these compounds.

In addition, the invention relates to the use of these compounds for the production of medicaments for the treatment of dyslipidaemia, atherosclerosis and diabetes.

The chronic effect of a calorie imbalance has resulted in an epidemic increase in the incidence of metabolic diseases in modern society. As a result, the World Health Organization has estimated that the global incidence of type 2 diabetes will exceed 300 million in 2030. Although several therapeutic options exist, none of them reverses the progress of this plague.

Although the control of glycated haemoglobin and plasmatic glycaemia in the fasted state are still considered as the primary objectives of antidiabetic treatments, acknowledgement of the fact that the diabetic state encompasses a range of metabolic disorders has broadened scope and expectations of future therapies. In the course of the last decade, hyperglycaemia has been shown to be not the only component of a series of anomalies affecting type-2 diabetic patients. Concurrent diseases, including insulin resistance, obesity, hypertension and dyslipidaemia, which, if they are present together or in part, constitutes what has been described as metabolic syndrome or syndrome X. This array of metabolic disorders forms the bases of a substantial increase in the incidence of cardiovascular disease in these patients.

In the search for novel and improved treatment options for diabetic patients, the family of receptors activated by the peroxisome proliferators (“peroxisome proliferator-activated receptor”: PPAR) appears potentially to be an ideal target. This family of ligand-activated transcription factors modulates numerous aspects of lipid and carbohydrate metabolism, thus having the possibility of attacking several facets of the diabetic phenotype. There are three types of PPAR: PPAR alpha, gamma and delta (PPARα, PPARγ and PPARδ, respectively).

PPARα is involved in stimulating the β-oxidation of fatty acids. In rodents, a change transmitted by a PPARα in the expression of genes involved in fatty acid metabolism is the basis of the phenomenon of peroxisome proliferation, a pleiotropic cellular response, mainly limited to the liver and the kidneys, which can lead to hepatocarcinogenesis in rodents. The phenomenon of peroxisome proliferation is not encountered in man. In addition to its role in peroxisome proliferation in rodents, PPARα is also involved in controlling the levels of HDL cholesterol in rodents and humans. This effect is at least partially based on a transcription regulation transmitted by a PPARα of the major HDL apolipoproteins, apo A-I and apo A-II. The hypotriglyceridaemiant action of fibrates and fatty acids also involves PPARα and can be summarised as follows: (i) increased lipolysis and clearance of the remaining particles, due to changes in the levels of lipoprotein lipase and of apo C-III, (ii) stimulation of fatty acid uptake by the cell and its subsequent conversion into acyl-CoA derivatives by induction of a protein for binding fatty acids and acyl-CoA synthase, (iii) induction of the β-oxidation pathways of fatty acids, (iv) reduction in the synthesis of fatty acids and triglycerides, and finally (v) reduction in the production of VLDL. As a result, both the improved catabolism of the triglyceride-rich particles and the reduced secretion of VLDL particles constitute mechanisms that contribute towards the hypolipidaemiant effect of fibrates.

Fibric acid derivatives, such as clofibrate, fenofibrate, benzafibrate, ciprofibrate, beclofibrate and etofibrate, and also gemfibrozil, each of which are PPARα ligands and/or activators, produce a substantial reduction in plasmatic triglycerides and also a certain increase in HDLs. The effects on LDL cholesterol are contradictory and may depend on the compound and/or the dyslipidaemic phenotype. For these reasons, this class of compounds was first used for the treatment of hypertriglyceridaemia (i.e. Fredrickson Type IV and V) and/or mixed hyperlipidaemia.

The activation of a PPARδ was initially reported as not being involved in the modulation of the levels of glucose or of triglycerides (Berger et al., J. Biol. Chem., (1999), Vol. 274, pp. 6718-6725). Later, it was shown that the activation of PPARδ leads to higher levels of HDL cholesterol in dbidb mice (Leibowitz et al., FEBS Letters, (2000), 473, 333-336). Furthermore, a PPARδ agonist, during its administration to obese adult insulin-resistant rhesus monkeys, caused a dramatic dose-dependent increase in HDL cholesterol in the serum, while at the same time reducing the levels of low-density LDLs, by depleting the triglycerides and the insulin (Oliver et al., PNAS, (2001), 98, 5306-5311). The same publication also showed that the activation of PPARδ increased the AI cassette binding the ATP inverse transporter of cholesterol and induced a flow of cholesterol specific for apolipoprotein A1. Taken together, these observations suggest that the activation of PPARδ is useful for the treatment of and preventing diseases and cardiovascular states comprising atherosclerosis, hypertriglyceridaemia and mixed dyslipidaemia (PCT publication WO 01/00603 (Chao et al.)).

The subtypes of PPARγ receptor are involved in the activation of the programme of adipocyte differentiation and are not involved in the stimulation of peroxisome proliferation in the liver. There are two known isoforms of PPARγ protein: PPARγ1 and PPARγ2, which differ only in the fact that PPARγ2 contains 28 additional amino acids at the amino end. The DNA sequences for the human isotypes are described by Elbrecht et al., BBRC, 224, (1996), 431-437. In mice, PPARγ2 is specifically expressed in the fat cells. Tontonoz et al., Cell, 79, (1994), 1147-1156, provide proof showing that one physiological role of PPARγ2 is to induce adipocyte differentiation. As with other members of the superfamily of nuclear hormone receptors, PPARγ2 regulates the expression of genes via an interaction with other proteins and binding to hormone response elements, for example in the 5′ lateral regions of the response genes. An example of a PPARγ2 response gene is the tissue-specific P2 adipocyte gene. Although peroxisome proliferators, comprising fibrates and fatty acids, activate the transcriptional activity of PPAR receptors, only prostaglandin J₂ derivatives have been identified as potential natural ligands of the PPARγ subtype, which also binds antidiabetic thiazolidinedione agents with high affinity.

It is generally thought that glitazones exert their effects by binding to receptors of the family of peroxisome proliferator-activated receptors (PPAR), by controlling certain transcription elements in relation with the biological species listed above. See Hulin et al., Current Pharm. Design, (1996), 2, 85-102. In particular, PPARγ has been imputed as a major molecular target for the glitazone class of insulin sensitisers.

Many compounds of glitazone type, which are PPAR agonists, have been approved for use in the treatment of diabetes. These are troglitazone, rosiglitazone and pioglitazone, which are all primary or exclusive agonists of PPARγ.

This indicates that the search for compounds having varying degrees of PPARα, PPARγ and PPARδ activation might lead to the discovery of medicaments that efficiently reduce triglycerides and/or cholesterol and/or glucose, presenting great potential in the treatment of diseases, such as type 2 diabetes, dyslipidaemia, syndrome X (comprising metabolic syndrome, i.e. reduced glucose tolerance, insulin resistance, hypertriglyceridaemia and/or obesity), cardiovascular diseases (comprising atherosclerosis) and hypercholesterolaemia.

The combinations of the PPAR activities that have been studied the most extensively are the PPAR alpha plus gamma combination (dual agonists) with, especially, tesaglitazar, and also the alpha, gamma plus delta triple combination (PPARpan agonists).

Although glitazones are beneficial in the treatment of NIDDM, a number of serious unfavourable side effects associated with the use of these compounds have been found. The most serious of these was toxicity to the liver, which has resulted in a certain number of deaths. The most serious problems arose in the use of troglitazone, which has recently been removed from the market for toxicity reasons.

Besides the potential hepatic toxicity of glitazones, other deleterious effects have been associated with PPAR gamma full agonists, for instance weight gain, anaemia and oedema, which limit their use (rosiglitazone, pioglitazone).

On account of the problems that have been encountered with glitazones, researchers in many laboratories have studied classes of PPAR agonists that are not glitazones and do not contain 1,3-thiazolidinedione species, but which modulate the three known subtypes of PPAR, together or separately, to variable degrees (measured by intrinsic power, maximum breadth of functional response or spectrum of changes in gene expression).

Thus, recent studies (cf. WO 01/30343 and WO 02/08188) have revealed that certain compounds have PPAR agonist or partial agonist properties, which are useful in the treatment of type 2 diabetes with reduced side effects with respect to the heart weight and body weight.

The inventors have now discovered a novel class of compounds that are partial or full agonists of PPARγ, with differing degrees of PPARα and/or PPARΓ activity.

More specifically, the invention relates to compounds derived from the 6-phenylhex-5-enoic acid of the formula (1) below:

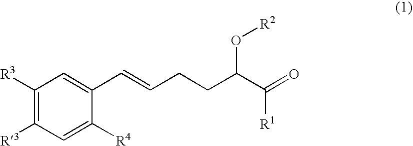

in which:

R¹ represents —O—R″¹ or —NR′¹R″¹, with R′¹ and R″¹, which may be identical or different, being chosen from a hydrogen atom, an alkyl radical, an alkenyl radical, an alkynyl radical, a cycloalkyl radical, an aryl radical and a heteroaryl radical;

R² is chosen from:
an alkyl, alkenyl or alkynyl radical;
an aryl radical, optionally substituted and/or optionally fused to a monocyclic or polycyclic, saturated or unsaturated 5- to 8-membered nucleus optionally containing one or more hetero atoms chosen from O, N and S, the said nucleus itself being optionally substituted, and
a saturated, unsaturated or aromatic, optionally substituted 5- to 8-membered monocyclic heterocyclic radical containing one or more hetero atoms chosen from O, N and S;

R³ is chosen from a hydrogen atom; a halogen atom chosen from chlorine, fluorine, bromine and iodine, preferably fluorine; an alkyl radical; an alkoxy radical; and an alkylcarbonyl radical;

or alternatively R³ forms with R′³, and with the carbon atoms bearing the substituents R³ and R′³, an optionally substituted, 5- or 6-membered carbocyclic radical;

R′³ represents a hydrogen atom or alternatively forms with R³ and with the carbon atoms bearing the substituents R³ and R′³, an optionally substituted 5- or 6-membered carbocyclic radical; and

R⁴ is chosen from a hydrogen atom, the hydroxyl radical and a radical —O—A—R⁵, in which

A represents a linear or branched alkylene chain containing from 1 to 6 carbon atoms; and

R⁵ is chosen from an optionally substituted aryl radical and an optionally substituted heterocyclic radical;

the possible optical isomers, oxide forms and solvates thereof, and also pharmaceutically acceptable addition salts thereof with acids or bases.

The acids that can be used for the formation of salts of compounds of the formula (1) are mineral or organic acids. The resulting salts are, for example, the hydrochlorides, hydrobromides, sulfates, hydrogen sulfates, dihydrogen phosphates, citrates, maleates, fumarates, trifluoroacetates, 2-naphthalenesulfonates and para-toluenesulfonates.

The bases that can be used for the formation of salts of compounds of the formula (1) are organic or mineral bases. The resulting salts are, for example, the salts formed with metals and especially alkali metals, alkaline-earth metals and transition metals (such as sodium, potassium, calcium, magnesium or aluminium) or with bases, for instance ammonia or secondary or tertiary amines (such as diethylamine, triethylamine, piperidine, piperazine or morpholine) or with basic amino acids, or with osamines (such as meglumine) or with amino alcohols (such as 3-aminobutanol and 2-amino-ethanol).

The invention especially encompasses the pharmaceutically acceptable salts, but also salts that allow a suitable separation or crystallisation of the compounds of the formula (1), such as the salts obtained with chiral amines or chiral acids.

Examples of chiral amines that can be used include quinine, brucine, (S)-1-(benzyloxymethyl)propylamine (3), (−)-ephedrine, (4S,5R)-(+)-1,2,3,4-tetramethyl-5-phenyl-1,3-oxazolidine, (R)-1-phenyl-2-p-tolylethyl-amine, (S)-phenylglycinol, (−)-N-methylephedrine, (+)-(2S,3R)-4-dimethyl-amino-3-methyl-1,2-diphenyl-2-butanol, (S)-phenylglycinol and (S)-α-methyl-benzylamine, or a mixture of two or more thereof.

Examples of chiral acids that can be used include (+)-d-di-O-ben-zoyltartaric acid, (−)-I-di-O-benzoyltartaric acid, (−)-di-O,O′-p-toluyl-l-tartaric acid, (+)-di-O,O′-p-toluyl-d-tartaric acid, (R)-(+)-malic acid, (S)-(−)-malic acid, (+)-camphanic acid, (−)-camphanic acid, R-(−)-1,1′-binaphthalene-2,2′-diyl hydrogen phosphate, (S)-(+)-1,1′-binaphthalene-2,2′-diyl hydrogen phosphate, (+)-camphoric acid, (−)-camphoric acid, (S)-(+)-2-phenylpropionic acid, (R)-(−)-2-phenylpropionic acid, d-(−)-mandelic acid, I-(+)-mandelic acid, d-tartaric acid and I-tartaric acid, or a mixture of two or more thereof.

The chiral acid is preferably chosen from (−)-di-O,O′-p-toluyl-I-tartaric acid, (+)-di-O,O′-p-toluyl-d-tartaric acid, (R)-(−)-1,1′-binaphthalene-2,2′-diyl hydrogen phosphate, (S)-(+)-1,1′-binaphthalene-2,2′-diyl hydrogen phosphate, d-tartaric acid and L-tartaric acid, or a mixture of two or more thereof.

The invention also encompasses the possible optical isomers, in particular stereoisomers and diastereoisomers, where appropriate, of the compounds of the formula (1), and also mixtures of the optical isomers in any proportions, including racemic mixtures.

Depending on the nature of the substituents, the compounds of the formula (1) may also be in various tautomeric forms, which are also included in the present invention, alone or as mixtures of two or more thereof, in all proportions.

The compounds of the formula (1) above also include the prodrugs of these compounds. The term “prodrugs” means compounds which, once administered to the patient, are chemically and/or biologically converted by the living body, into compounds of the formula (1).

In the compounds of the formula (1) defined above, unless specified otherwise, the term “alkyl radical” means a linear or branched hydrocarbon-based chain containing from 1 to 10 carbon atoms and better still from 1 to 6 carbon atoms, for example from 1 to 4 carbon atoms, optionally substituted by one or more substituents, which may be identical or different, chosen from halogen atoms and trifluoromethyl, trifluoromethoxy, hydroxyl, alkoxy, alkoxycarbonyl, carboxyl and oxo radicals.

Examples of preferred alkyl radicals are methyl, ethyl, propyl, isopropyl, butyl, isobutyl, tert-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 1-ethylbutyl, 1-methyl-1-ethylpropyl, heptyl, 1-methylhexyl, 1-propylbutyl, 4,4-dimethylpentyl, octyl, 1-methylheptyl, 2-methylhexyl, 5,5-dimethylhexyl, nonyl, decyl, 1-methylnonyl, 3,7-dimethyl-octyl and 7,7-dimethyloctyl.

The term “alkoxy radical” should be understood as an —O-alkyl radical, the term alkyl being as defined above.

The term “alkylcarbonyl radical” should be understood as a —C(═O)-alkyl radical, the term alkyl being as defined above.

The term “alkylene chain” means a divalent radical of linear or branched aliphatic hydrocarbon-based type derived from the alkyl groups defined above by abstraction of a hydrogen atom. Preferred examples of alkylene chains are —(CH₂)ₖ— chains in which k represents an integer chosen from 1, 2, 3, 4, 5 and 6, and the chains >CH(CH₃), >C(CH₃)₂, —CH₂—CH(CH₃)—CH₂— and —CH₂—C(CH₃)₂—CH₂—.

The term “alkenyl radical” means a linear or branched hydrocarbon-based chain containing from 2 to 10 carbon atoms, preferably from 2 to 8 carbon atoms and advantageously from 2 to 6 carbon atoms, containing one, two or more, unsaturations in the form of a double bond, the said chain being optionally substituted by one or more substituents, which may be identical or different, chosen from halogen atoms and trifluoromethyl, trifluoromethoxy, hydroxyl, alkoxy, alkoxycarbonyl, carboxyl and oxo radicals.

Examples of alkenyl radicals that may be mentioned include the ethylenyl radical, the propenyl radical, the isopropenyl radical, the but-2-enyl radical, pentenyl radicals and hexenyl radicals.

The term “alkynyl radical” means a linear or branched hydrocarbon-based chain containing from 2 to 10 carbon atoms, preferably from 2 to 8 carbon atoms and advantageously from 2 to 6 carbon atoms, containing one, two or more unsaturations in the form of a triple bond, the said chain being optionally substituted by one or more substituents, which may be identical or different, chosen from halogen atoms and trifluoromethyl, trifluoromethoxy, hydroxyl, alkoxy, alkoxycarbonyl, carboxyl and oxo radicals.

Examples of alkynyl radicals that may be mentioned include the ethynyl radical, the propynyl radical, the but-2-ynyl radical, pentynyl radicals and hexynyl radicals.

In the present invention, the cycloalkyl radical is taken to mean a cyclic hydrocarbon-based radical containing from 4 to 9 carbon atoms, preferably 5, 6 or 7 carbon atoms and advantageously 5 or 6 carbon atoms, optionally containing one or more unsaturations in the form of double and/or triple bonds, the said cycloalkyl radical being optionally substituted by one or more substituents, which may be identical or different, chosen from halogen atoms and alkyl, alkenyl, alkynyl, trifluoromethyl, trifluoromethoxy, hydroxyl, alkoxy, alkoxycarbonyl, carboxyl and oxo radicals.

Preferred examples of cycloalkyl radicals are cyclobutyl, cyclopentyl, cyclopentenyl, cyclohexyl, cyclohexenyl, cyclohexadienyl, cycloheptyl, cycloheptenyl and cycloheptadienyl.

The term “halogen atom” is taken to mean a chlorine, bromine, iodine or fluorine atom, preferably a fluorine or chlorine atom.

According to the invention, the term “aryl radical” means a monocyclic or polycyclic carbocyclic aromatic radical containing from 6 to 18 carbon atoms and preferably from 6 to 10 carbon atoms. Aryl radicals that may be mentioned include phenyl, naphthyl, anthryl and phenanthryl radicals.

The heterocyclic radicals are monocyclic, bicyclic or tricyclic radicals containing one or more hetero atoms generally chosen from O, S and N, optionally in oxidised form (in the case of S and N), and optionally one or more unsaturations in the form of double bonds. If they are totally saturated, the heterocyclic radicals are said to be aromatic or heteroaryl radicals.

Preferably, at least one of the monocycles constituting the heterocycle contains from 1 to 4 endocyclic hetero atoms and better still from 1 to 3 hetero atoms.

Preferably, the heterocycle consists of one or more monocycles, each of which is 5- to 8-membered.

Examples of 5- to 8-membered monocyclic aromatic heterocyclic radicals are the heteroaryl radicals derived, by abstraction of a hydrogen atom, from aromatic heterocycles, such as pyridine, furan, thiophene, pyrrole, imidazole, thiazole, isoxazole, isothiazole, furazane, pyridazine, pyrimidine, pyrazine, thiazines, oxazole, pyrazole, oxadiazole, triazole and thiadiazole.

Preferred aromatic heterocyclic radicals that may be mentioned include pyridyl, pyrimidinyl, triazolyl, thiadiazolyl, oxazolyl, thiazolyl and thienyl radicals.

Examples of bicyclic heteroaryls in which each monocycle is 5- to 8-membered are chosen from indolizine, indole, isoindole, benzofuran, benzothiophene, indazole, benzimidazole, benzothiazole, benzofurazane, benzothiofurazane, purine, quinoline, isoquinoline, cinnoline, phthalazine, quinazoline, quinoxaline, naphthyridines, pyrazolotriazines (such as pyrazolo-1,3,4-triazine), pyrazolopyrimidine and pteridine.

Preferred heteroaryl radicals that may be mentioned include the quinolyl, pyridyl, benzothiazolyl and triazolyl radicals.

The tricyclic heteroaryls in which each monocycle is 5- to 8-membered are chosen, for example, from acridine, phenazine and carbazole.

Saturated or unsaturated, 5- to 8-membered monocyclic heterocycles are the saturated or, respectively, unsaturated derivatives of the aromatic heterocycles mentioned above.

More particularly, mention may be made of morpholine, piperidine, thiazolidine, oxazolidine, tetrahydrothienyl, tetrahydrofuryl, pyrrolidine, isoxazolidine, imidazolidine and pyrazolidine.

When R³ and R′³ together form a carbocycle with the carbon atoms that bear them, it is preferable for R³, R′³ and the carbons to which they are attached to form a 5-, 6- or 7-membered ring, preferably a 5- or 6-membered ring and more preferably a 5-membered ring.

The said carbocycle may optionally be substituted with one or more substituents, which may be identical or different, chosen from a halogen atom, a trifluoromethyl, trifluoromethoxy or hydroxyl radical, and an alkyl, alkoxy, alkoxycarbonyl, carboxyl or oxo radical.

It is in particular preferable for R³ and R′³ to form, together with the carbon atoms that bear them, a 5-membered carbocycle substituted by an oxo group.

The aryl and heterocyclic radicals are optionally substituted by one or more of the following radicals G:

trifluoromethyl; trifluoromethoxy; styryl; halogen atom; monocyclic, bicyclic or tricyclic aromatic heterocyclic radical containing one or more hetero atoms chosen from O, N and S; and optionally substituted by one or more radicals T as defined below; a Het-CO— group, in which Het represents an aromatic heterocyclic radical as defined above, optionally substituted by one or more radicals T; a C₁-C₆ alkylenediyl chain; a C₁-C₆ alkylenedioxy chain; nitro; cyano; (C₁-C₁₀)alkyl; (C₁-C₁₀)alkylcarbonyl; (C₁-C₁₀)alkoxy-carbonyl-A- in which A represents (C₁-C₆)alkylene, (C₂-C₆)alkenylene or a bond; (C₃-C₁₀)cycloalkyl; trifluoromethoxy; di(C₁-C₁₀)alkylamino; (C₁-C₁₀)—alkoxy(C₁-C₁₀)alkyl; (C₁-C₁₀)alkoxy; (C₆-C₁₈)aryl optionally substituted by one or more radicals T; (C₆-C₁₈)aryl(C₁-C₁₀)alkoxy(CO)ₙ— in which n is 0 or 1 and aryl is optionally substituted by one or more radicals T; (C₆-C₁₈)aryloxy-(CO)ₙ— in which n is 0 or 1 and aryl is optionally substituted by one or more radicals T; (C₆-C₁₈)arylthio in which aryl is optionally substituted by one or more radicals T; (C₆-C₁₈)aryloxy(C₁-C₁₀)alkyl(CO)ₙ— in which n is 0 or 1 and aryl is optionally substituted by one or more radicals T; a saturated or unsaturated, 5- to 8-membered monocyclic heterocycle containing one or more hetero atoms chosen from O, N and S, optionally substituted by one or more radicals T; (C₆-C₁₈)arylcarbonyl optionally substituted by one or more radicals T; (C₆-C₁₈)arylcarbonyl-B—(CO)ₙ— in which n is 0 or 1; B represents (C₁-C₆)—alkylene or (C₂-C₆)alkenylene and aryl is optionally substituted by one or more radicals T; (C₆-C₁₈)aryl-C—(CO)ₙ— in which n is 0 or 1, C represents (C₁-C₆)alkylene or (C₂-C₆)alkenylene and aryl is optionally substituted by one or more radicals T; (C₆-C₁₈)aryl fused with a saturated or unsaturated heterocycle as defined above, optionally substituted by one or more radicals T; (C₂-C₁₀)alkynyl. T is chosen from a halogen atom; (C₆-C₁₈)aryl; (C₁-C₆)alkyl; (C₁-C₆)alkoxy; (C₁-C₆)alkoxy(C₆-C₁₈)aryl; nitro; carboxyl; (C₁-C₆)alkoxycarboxyl; and T may represent oxo if it substitutes a saturated or unsaturated heterocycle; or alternatively T represents (C₁-C₆)alkoxycarbonyl(C₁-C₆)alkyl; or (C₁-C₆)alkylcarbonyl((C₁-C₆)alkyl)ₙ— in which n is 0 or 1.

Among the compounds of the formula (1), the ones that are preferred are those for which R¹ represents —O—R″¹ and most particularly those for which R¹ represents —O—R′¹, R′¹ being a hydrogen atom or an alkyl radical.

A first preferred group of compounds of the invention consists of compounds having one or more of the following characteristics, taken separately or as a combination of one, several or all of them:

R¹ represents —O—R′¹, R′¹ being chosen from a hydrogen atom, an alkyl radical, an alkenyl radical, an alkynyl radical, a cycloalkyl radical, an aryl radical and a heteroaryl radical;

R² represents an alkyl radical or an optionally substituted aryl radical;

R³ is chosen from a hydrogen atom, a halogen atom chosen from chlorine, fluorine, bromine and iodine, preferably fluorine; an alkyl radical; an alkoxy radical; and an alkylcarbonyl radical;

or alternatively R³ forms with R′³ and with the carbon atoms bearing the substituents R³ and R′³, an optionally substituted 5- or 6-membered carbocyclic radical;

R′³ represents a hydrogen atom or forms with R³, and with the carbon atoms bearing the substituents R³ and R′³, an optionally substituted 5- or 6-membered carbocyclic radical; and

R⁴ is chosen from a hydrogen atom, a hydroxyl radical and a radical —O-A-R⁵, in which

A represents a linear or branched alkylene chain containing from 1 to 6 carbon atoms; and

R⁵ is chosen from an optionally substituted aryl radical and an optionally substituted heterocyclic radical;

the possible optical isomers, oxide forms and solvates thereof, and also the pharmaceutically acceptable addition salts thereof with acids or bases.

Another even more preferred group of compounds of the invention consists of compounds having one or more of the following characteristics, taken separately or as a combination of one, several or all of them:

R¹ represents —O—R′¹, R′¹ being chosen from a hydrogen atom, an alkyl radical, a cycloalkyl radical, an aryl radical and a heteroaryl radical;

R² represents a C₁-C₆ alkyl radical or an optionally substituted phenyl radical;

R³ is chosen from a hydrogen atom, a halogen atom chosen from chlorine, fluorine, bromine and iodine, preferably fluorine; a C₁-C₆ alkyl radical; a C₁-C₆ alkoxy radical; and a C₁-C₆ alkylcarbonyl radical;

or alternatively R³ forms with R′³ and with the carbon atoms bearing the substituents R³ and R′³, an optionally substituted 5-membered carbocyclic radical;

R′³ represents a hydrogen atom or forms with R³, and with the carbon atoms bearing the substituents R³ and R′³, an optionally substituted 5-membered carbocyclic radical; and

R⁴ is chosen from a hydrogen atom, a hydroxyl radical and a radical —O-A-R⁵, in which

A represents a linear alkylene chain corresponding to the formula —(CH₂)ₖ— in which k represents an integer chosen from 1, 2, 3, 4, 5 and 6; and

R⁵ is chosen from an optionally substituted phenyl radical and an optionally substituted heterocyclic radical;

the possible optical isomers, oxide forms and solvates thereof, and also the pharmaceutically acceptable addition salts thereof with acids or bases.

Another preferred group of compounds of the invention consists of compounds having one or more of the following characteristics, taken separately or as a combination of one, several or all of them:

R¹ represents —O—R′¹, R′¹ being chosen from a hydrogen atom and a ethyl, ethyl, propyl or isopropyl radical;

R² represents a methyl, ethyl, propyl or isopropyl radical, or a subtituted phenyl radical;

R³ is chosen from a hydrogen atom, a fluorine atom, a methyl, ethyl, ropyl or isopropyl radical, a methoxy, ethoxy, propoxy or isopropoxy radical, nd a methylcarbonyl, ethylcarbonyl or propylcarbonyl radical;

or alternatively R³ forms with R′³, and with the carbon atoms bearing he substituents R³ and R′³, a substituted 5-membered carbocyclic radical;

R′³ represents a hydrogen atom or forms with R³, and with the carbon atoms bearing the substituents R³ and R′³, a substituted 5-membered carbocyclic radical; and

R⁴ is chosen from a hydrogen atom, a hydroxyl radical and a radical —O-A-R⁵, in which

A represents a linear alkylene chain corresponding to the formula —(CH₂)ₖ— in which k represents an integer chosen from 1, 2 and 3; and

R⁵ is chosen from a substituted phenyl radical and a substituted 5- or 6-membered heterocyclic radical;

the possible optical isomers, oxide forms and solvates thereof, and also the pharmaceutically acceptable addition salts thereof with acids or bases.

Another even more preferred group of compounds of the invention consists of compounds having one or more of the following characteristics, taken separately or as a combination of one, several or all of them:

R¹ represents —O—R′¹, R′¹ being chosen from a hydrogen atom, a methyl radical and an ethyl radical;

R² represents a methyl or ethyl radical, or a substituted phenyl radical;

R³ is chosen from a hydrogen atom, a fluorine atom, a methyl or ethyl radical, a methoxy or ethoxy radical, and a methylcarbonyl or ethylcarbonyl radical;

or alternatively R³ forms with R′³, and with the carbon atoms bearing the substituents R³ and R′³, a saturated 5-membered carbocyclic radical substituted by an oxo group;

R′³ represents a hydrogen atom or forms with R³, and with the carbon atoms bearing the substituents R³ and R′³, a saturated 5-membered carbocyclic radical substituted by an oxo group; and

R⁴ is chosen from a hydrogen atom, a hydroxyl radical and a radical —O-A-R⁵, in which

A represents an alkylene chain corresponding to the formula —(CH₂)ₖ— in which k represents an integer chosen from 1 and 2; and

R⁵ is chosen from a substituted phenyl radical and a substituted 5- or 6-membered heterocyclic radical comprising not more than three hetero atoms and preferably not more than two hetero atoms, chosen from O, N and S;

the possible optical isomers, oxide forms and solvates thereof, and the pharmaceutically acceptable addition salts thereof with acids or bases.

The substituents on the aryl and heterocyclic radicals are preferably chosen from methyl, ethyl, methoxy, phenyl, fluorine and trifluoromethyl.

The heterocyclic radicals are preferentially chosen from thienyl, benzothiophenyl, pyridyl and oxazolyl radicals.

The compounds of the formula (1) that are more particularly preferred are those chosen from:
6-[5-methoxy-2-(5-methyl-2-phenyloxazol-4-ylmethoxy)phenyl]-2-(4-trifluoromethylphenoxy)hex-5-enoic acid;
ethyl 2-(4′-fluorobiphenyl-4-yloxy)-6-[5-methoxy-2-(5-methyl-2-phenyl-oxazol-4-ylmethoxy)phenyl]hex-5-enoate;
6-{5-methoxy-2-[2-(5-methyl-2-phenyloxazol-4-yl)ethoxy]phenyl}-2-(4-trifluoromethylphenoxy)hex-5-enoic acid;
2-(4′-fluorobiphenyl-4-yloxy)-6-[5-methoxy-2-(4-trifluoromethylbenzyloxy)-phenyl]hex-5-enoic acid;
6-[2-(2-fluorobenzyloxy)-5-methoxyphenyl]-2-(4′-fluorobiphenyl-4-yloxy)-hex-5-enoic acid;
2-ethoxy-6-{5-fluoro-2-[2-(5-methyl-2-phenyloxazol-4-yl)ethoxy]phenyl}-hex-5-enoic acid;
6-{2-[2-(5-ethylpyridin-2-yl)ethoxy]-5-methoxyphenyl}-2-(2-methoxy-phenoxy)hex-5-enoic acid;
6-{2-[2-(5-ethylpyridin-2-yl)ethoxy]-5-fluorophenyl}-2-(2-methoxyphen-oxy)hex-5-enoic acid;
ethyl 6-{5-fluoro-2-[2-(5-methyl-2-phenyloxazol-4-yl)ethoxy]phenyl}-2-(4-trifluoromethylphenoxy)hex-5-enoate;
6-{5-fluoro-2-[2-(5-methyl-2-phenyloxazol-4-yl)ethoxy]phenyl}-2-(4-trifluoromethylphenoxy)hex-5-enoic acid;
6-{5-Methoxy-2-[2-(5-methyl-2-phenyloxazol-4-yl)ethoxy]phenyl}-2-(2-methoxyphenoxy)hex-5-enoic acid;
6-[2-(2-fluorobenzyloxy)-5-methoxyphenyl]-2-(2-methoxyphenoxy)hex-5-enoic acid;
ethyl 6-[5-methoxy-2-(5-methyl-2-phenyloxazol-4-ylmethoxy)phenyl]-2-(4-trifluoromethylphenoxy)hex-5-enoate;
2-ethoxy-6-{2-[2-(5-ethylpyridin-2-yl)ethoxy]-5-fluorophenyl}hex-5-enoic acid;
2-(2-methoxyphenoxy)-6-[5-methoxy-2-(4-trifluoromethylbenzyloxy)-phenyl]hex-5-enoic acid;
6-{5-fluoro-2-[2-(5-methyl-2-phenyloxazol-4-yl)ethoxy]phenyl}-2-(2-methoxyphenoxy)hex-5-enoic acid;
6-{2-[2-(5-ethylpyridin-2-yl)ethoxy]-5-methylphenyl}-2-(2-methoxy-phenoxy)hex-5-enoic acid;
6-{5-acetyl-2-[2-(5-methyl-2-phenyloxazol-4-yl)ethoxy]phenyl}-2-(4′-fluorobiphenyl-4-yloxy)hex-5-enoic acid;
6-{5-acetyl-2-[2-(5-methyl-2-phenyl-oxazol-4-yl)ethoxy]phenyl}-2-(2-methoxyphenoxy)hex-5-enoic acid;
6-{2-[2-(5-ethylpyridin-2-yl)ethoxy]-5-fluorophenyl)-2-(4-trifluoromethyl-phenoxy)hex-5-enoic acid;
6-[5-methoxy-2-(5-methyl-2-phenyloxazol-4-ylmethoxy)phenyl]-2-(2-methoxyphenoxy)hex-5-enoic acid;
ethyl 2-ethoxy-6-[5-methoxy-2-(4-trifluoromethylbenzyloxy)phenyl]hex-5-enoate;
2-ethoxy-6-[5-methoxy-2-(4-trifluoromethylbenzyloxy)phenyl]hex-5-enoic acid;
2-ethoxy-6-[2-(2-fluorobenzyloxy)-5-methoxyphenyl]hex-5-enoic acid;
2-(4′-fluorobiphenyl-4-yloxy)-6-[5-methoxy-2-(5-methyl-2-phenyloxazol-4-ylmethoxy)phenyl]hex-5-enoic acid;
2-ethoxy-6-{5-methoxy-2-[2-(5-methyl-2-phenyloxazol-4-yl)ethoxy]-phenyl}hex-5-enoic acid;
2-ethoxy-6-[5-methoxy-2-(5-methyl-2-phenyloxazol-4-ylmethoxy)-phenyl]hex-5-enoic acid;
2-ethoxy-6-{2-[2-(5-ethylpyridin-2-yl)ethoxy]-5-methylphenyl}-hex-5-enoic acid;
ethyl 2-ethoxy-6-{5-methoxy-2-[2-(5-methyl-2-phenyloxazol-4-yl)eth-oxy]phenyl}hex-5-enoate;
2-[4-(5-chlorothiophen-2-yl)phenoxy]-6-phenylhex-5-enoic acid;
ethyl 2-(4′-fluorobiphenyl-4-yloxy)-6-[6-(5-methyl-2-phenyloxazol-4-yl-methoxy)-1-oxoindan-5-yl]hex-5-enoate;
2-(4′-fluorobiphenyl-4-yloxy)-6-[6-(5-methyl-2-phenyloxazol-4-ylmeth-oxy)-1-oxoindan-5-yl]hex-5-enoic acid;
ethyl 2-(2-methoxyphenoxy)-6-[6-(5-methyl-2-phenyloxazol-4-ylmeth-oxy)-1-oxoindan-5-yl]hex-5-enoate; and
2-(2-methoxyphenoxy)-6-[6-(5-methyl-2-phenyloxazol-4-ylmethoxy)-1-oxoindan-5-yl]hex-5-enoic acid

and from the possible optical isomers, oxide forms and solvates, and also the pharmaceutically acceptable addition salts with acids or bases, of these compounds.

The invention also relates to pharmaceutical compositions comprising a pharmaceutically effective amount of at least one compound of the formula (1) as defined above in combination with one or more pharmaceutically acceptable vehicles.

These compositions can be administered orally in the form of tablets, gel capsules or granules with immediate release or controlled release, intravenously in the form of an injectable solution, transdermally in the form of an adhesive transdermal device, or locally in the form of a solution, cream or gel.

A solid composition for oral administration is prepared by adding to the active principle a filler and, where appropriate, a binder, a disintegrant, a lubricant, a dye or a flavour enhancer, and by forming the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatine, shellac, hydroxypropylcellulose, hydroxypropylmethylcellulose, calcium citrate, dextrin and pectin. Examples of lubricants include magnesium stearate, talc, polyethylene glycol, silica and hardened plant oils. The dye can be any dye permitted for use in medicaments. Examples of flavour enhancers include cocoa powder, mint in herb form, aromatic powder, mint in oil form, borneol and cinnamon powder. Needless to say, the tablet or granule may be appropriately coated with sugar, gelatine or the like.

An injectable form comprising the compound of the present invention as active principle is prepared, where appropriate, by mixing the said compound with a pH regulator, a buffer, a suspending agent, a solubilising agent, a stabiliser, a tonicity agent and/or a preserving agent, and by converting the mixture into a form for intravenous, subcutaneous or intramuscular injection according to a standard process. Where appropriate, the injectable form obtained can be freeze-dried via a standard process.

Examples of suspending agents include methylcellulose, polysorbate 80, hydroxyethylcellulose, acacia, powdered gum tragacanth, sodium carboxymethyl cellulose and polyethoxylated sorbitan monolaurate.

Examples of solubilising agents include castor oil solidified with polyoxyethylene, polysorbate 80, nicotinamide, polyethoxylated sorbitan monolaurate and the ethyl ester of castor oil fatty acid.

In addition, the stabiliser encompasses sodium sulfite, sodium metasulfite and ether, while the preserving agent encompasses methyl p-hydroxybenzoate, ethyl p-hydroxybenzoate, sorbic acid, phenol, cresol and chlorocresol.

The present invention also relates to the use of a compound of the formula (1) of the invention for the preparation of a medicament for the prevention or treatment dyslipidaemia, atherosclerosis and diabetes.

The effective administration doses and posologies of the compounds of the invention, intended for the prevention or treatment of a disease, condition or state caused by or associated with modulation of the activity of the PPARs, depends on a large number of factors, for example on the nature of the agonist, the size of the patient, the desired aim of the treatment, the nature of the pathology to be treated, the specific pharmaceutical composition used and the observations and conclusions of the treating doctor.

For example, in the case of an oral administration, for example, a tablet or a gel capsule, a possible suitable dosage of the compounds of the formula (1) is between about 0.1 mg/kg and about 100 mg/kg of body weight per day, preferably between about 0.5 mg/kg and about 50 mg/kg of body weight per day, more preferentially between about 1 mg/kg and about 10 mg/kg of body weight per day and more preferably between about 2 mg/kg and about 5 mg/kg of body weight per day of active material.

If representative body weights of 10 kg and 100 kg are considered in order to illustrate the daily oral dosage range that can be used and as described above, suitable dosages of the compounds of the formula (1) will be between about 1-10 mg and 1000-10 000 mg per day, preferably between about 5-50 mg and 500-5000 mg per day, more preferably between about 10.0-100.0 mg and 100.0-1000.0 mg per day and even more preferentially between about 20.0-200.0 mg and about 50.0-500.0 mg per day of active material comprising a preferred compound.

These dosage ranges represent total amounts of active material per day for a given patient. The number of administrations per day at which a dose is administered may vary within wide proportions as a function of pharmacokinetic and pharmacological factors, such as the half-life of the active material, which reflects its rate of catabolism and of clearance, and also the minimum and optimum levels of the said active material reached in the blood plasma or other bodily fluids of the patient and which are required for therapeutic efficacy.

Many other factors should also be considered in deciding upon the number of daily administrations and the amount of active material that should be administered at a time. Among these other factors, and not the least of which, is the individual response of the patient to be treated.

The present invention also relates to a general process for the preparation of the compounds of the formula (1), by coupling a compound of the formula (2) with a hex-5-enoic acid derivative of the formula (3):

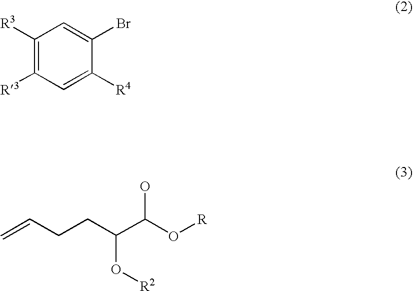

in which formulae R², R³, R′³ and R⁴ are as defined above for the formula (1) and R represents a protecting group for the acid function, for example an alkyl radical, such as methyl or ethyl,

the said coupling being performed by catalysis, for example using a palladium catalyst, in particular dichlorobis(tri-ortho-tolylphosphine)palladium II, in the presence of a phosphine, for example tri-ortho-tolylphosphine, in the presence of a weak base, such as an organic base, for example triethylamine, in polar medium, for example with a solvent, such as dimethylformamide, the said reaction preferably being performed at elevated temperature, for example at about 110° C., for a time ranging from one to several hours, for example from about 5 hours to about 7 hours, so as to obtain the compound of the formula (1_{R}):

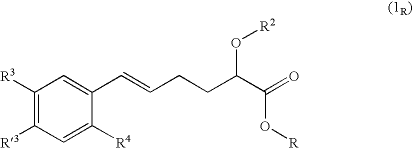

in which R², R³, R′³ and R⁴ are as defined above for the formula (1) and R represents the protecting group of the acid function defined above,

which compound of the formula (1_{R}) is converted, according to standard techniques known to those skilled in the art, into the corresponding acid of the formula (1_{OH}):

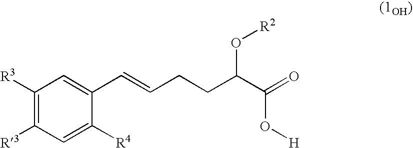

which is a special case of the compounds of the formula (1) in which R¹ represents a hydroxyl radical,

and the acid is optionally esterified, or converted into the corresponding amide, also according to standard techniques, to give the set of compounds of the formula (1) with R¹ other than a hydroxyl radical.

This synthetic method applies to all the compounds of the formula (1) according to the present invention, and is described in greater detail in the synthesis of the compounds of Examples 1 and 3 below, for the coupling reaction, and in the synthesis of the compounds of Examples 2 and 4 below, for the deprotection reaction (saponification).

It should be understood that the compounds of the formula (1_{R}) above, if R represents an alkyl radical, form part of the compounds of the formula (1) according to the present invention (R¹═—O—R′¹, with R′¹═alkyl).

If such compounds are desired, the steps of deprotection of the acid function and then of esterification are superfluous.

According to one variant, the compounds of the formula (1) can also be prepared by coupling a compound of the formula (3) with a compound of the formula (2^{OH}):

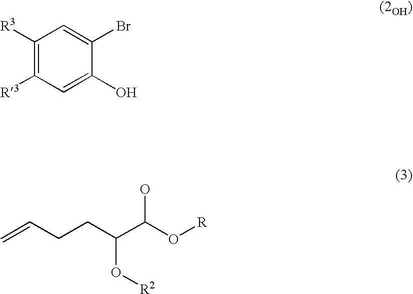

which is a special case of the compounds of the formula (2) defined above in which R⁴ represents a hydroxyl radical,

under coupling conditions that are similar, or even identical, to those described above, to give the compound of the formula (1′_{OH}):

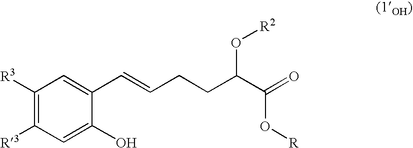

in which R, R², R³ and R′³ are as defined above,

the hydroxyl function borne by the phenyl nucleus being optionally converted into the various substituents defined for R⁴ in the formula (1), the group R then being optionally removed to give the acid function, and consequently the compounds of the formula (1_{OH}), which are finally optionally esterified, or converted into the corresponding amides, to form the set of compounds of the formula (1), with R¹ other than a hydroxyl radical.

This method is more particularly detailed in the preparation of the compound of Example 5.

The reactions for converting the hydroxyl function borne by the phenyl nucleus into the various substituents defined for R⁴ are known and are readily accessible to those skilled in the art.

By way of illustration, the compound of the formula (1′_{OH}) defined above can be reacted with a compound of the formula (4):

X-A-R⁵ (4)

in which X represents OH or a halogen atom, and A and R⁵ are as defined above for the compounds of the formula (1).

If X represents —OH, this reaction is preferably performed in a polar aprotic solvent, such as a linear or cyclic ether, such as diethyl ether, di- tert-butyl ether, diisopropyl ether or dimethoxyethane, or, such as dioxane or tetrahydrofuran, tetrahydrofuran and dimethoxyethane being preferred.

According to one preferred embodiment of the invention, the molar ratio of the compound of the formula (1′_{OH}) to the alcohol HO-A-R⁵ ranges between 0.9 and 1.5, an approximately stoichiometric ratio of between 0.9 and 1.3 and preferably between 0.9 and 1.1 being desirable.

So as to facilitate the reaction, it is desirable to add to the medium a coupling agent, such as a lower alkyl (i.e. a C₁-C₆ alkyl) azodicarboxylate, for example diisopropyl azodicarboxylate.

If it is present in the reaction medium, the coupling agent is incorporated into the medium in a proportion of from 1 to 5 equivalents and better still from 1 to 3 equivalents, for example in a proportion of 1 to 2 molar equivalents relative to the initial amount of compound of the formula (1′_{OH}).

Preferably, it is also recommended to introduce a phosphine into the reaction medium, such as triphenylphosphine. In this case, the molar ratio of triphenylphosphine to the compound of the formula (1′_{OH}) is preferably maintained between 1 and 5, for example between 1 and 3 and especially between 1 and 2.

In the formula (4), if X represents a halogen atom, the solvent is preferably of ketone type and a base is introduced into the reaction medium, preferably a mineral base chosen from sodium hydroxide, potassium hydroxide, sodium hydrogen carbonate, potassium hydrogen carbonate, sodium carbonate and potassium carbonate.

Usually, the molar ratio of the base to the compound of the formula (1′_{OH}) ranges between 1 and 5 and better still between 1 and 3.

If X represents a halogen atom, the reaction temperature generally ranges between 10° C. and 120° C., for example between 60° C. and 100° C. and better still between 70° C. and 90° C.

If X represents —OH, the reaction temperature generally ranges between −15° C. and 50° C., it being understood that temperatures of between −15° C. and 10° C. are desirable in the presence of a coupling agent.

The compound of Example 6 was prepared according to this method, the details of which are given in a non-limiting manner in this example.

The compounds of the formula (1) for which R³, R′³ and R⁴ each represent a hydrogen atom can advantageously be prepared by reacting a compound of the formula (5):

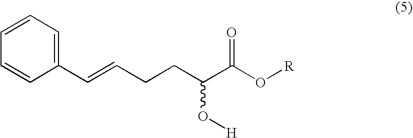

in which R is as defined above,

with a compound of the formula (6):

R²—X (6),

in which R² is as defined for the compound of the formula (1), and X represents an —OH radical or a halogen atom,

according to standard techniques known to those skilled in the art, for example according to the method described above for coupling between the compound of the formula (1′_{OH}) and the compound of the formula (4),

The compounds thus obtained correspond to the formula (1_{R}) defined above, with R³, R′³ and R⁴ each representing a hydrogen atom.

The details of an illustrative example of this preparation method are given in Example 8.

Depending on the nature of the substituents, a person skilled in the art will understand that it may prove necessary to perform the coupling reaction, for example of the compound of the formula (5), with a compound R′²—X, in which R′² is a precursor of the substituent R², the completion of the formation of the desired group R² being performed after the actual coupling reaction. An example of such a synthetic route is illustrated in Example 10.

The compounds of the formula (1) in which R¹ represents OH can advantageously be obtained by saponification of the corresponding compounds of the formula (1) in which R¹ represents an alkoxy radical, or alternatively starting with the compounds of the formula (1_{R}), in which R represents an alkyl radical. The saponification can be performed via the action of a base, such as a mineral base chosen from lithium hydroxide, potassium hydroxide, sodium hydroxide, sodium hydrogen carbonate, potassium hydrogen carbonate, sodium carbonate and potassium carbonate. The molar amount of base to be used generally ranges from 1 to 20 equivalents and preferably from 1 to 12 equivalents depending on the strength of the selected base.

The reaction is preferably performed in a solvent of polar protic type and more preferably in a mixture of a lower (C₁-C₄) alkanol and water, such as a mixture of ethanol and water or methanol and water.

The reaction temperature advantageously ranges between 35° and 120° C. and better still between 40° and 100° C., for example between 50° C. and reflux.

In the processes described above, it should be understood that the operating conditions may vary substantially as a function of the various substituents present in the compounds of the formula (1) that it is desired to prepare. Such variations and adaptations are readily accessible to those skilled in the art, for example from scientific reviews, the patent literature, Chemical Abstracts, and computer databases, including the Internet. Similarly, the starting materials are either commercially available or accessible via syntheses that a person skilled in the art can readily find, for example in the various publications and databases described above.

The optical isomers of the compounds of the formula (1) can be obtained on the one hand via standard techniques for separating and/or purifying isomers known to those skilled in the art, starting with the racemic mixture of the compound of the formula (1). The optical isomers can also be obtained directly via stereoselective synthesis of an optically active starting compound, or via separation or recrystallisation of the optically active salts of the compounds of the formula (1), the salts being obtained with chiral amines or chiral acids.

The examples that follow illustrate the present invention without limiting it in any way. In these examples and the proton nuclear magnetic resonance data (300 MHz NMR), the following abbreviations have been used: s for singlet, d for doublet, t for triplet, q for quartet, o for octet and m for complex multiplet. The chemical shifts δ are expressed in ppm.

### EXAMPLES

### Example 1

### Ethyl 2-(4′-fluorobiphenyl-4-yloxy)-6-[5-methoxy-2-(4-trifluoro-methylbenzyloxy)phenyl]hex-5-enoate

Step 1

A mixture of 2-bromo-4-methoxyphenol (2.0 g; 10 mmol), 4-tri-fluoromethylbenzyl alcohol (1.5 ml; 11 mmol) and triphenylphosphine (2.84 g; 11 mmol) in toluene (40 ml) is heated to 54° C. A solution of diisopropyl azo-dicarboxylate (DIAD) (1.9 ml; 10 mmol) in toluene is then added dropwise. The mixture is heated for one hour at 54° C. and then stirred overnight at room temperature. The medium is concentrated (9.5 g) and then purified by flash chromatography (95/5 heptane/ethyl acetate). An oil (2.35 g; 65%) that crystallises on standing is obtained.

¹H NMR (300 MHz, chloroform-D), δ ppm: 3.9 (s, 3 H); 5.3 (s, 2 H); 6.9 (dd, J=8.9, 2.8 Hz, 1 H); 7.0 (m, 1 H); 7.3 (d, J=2.8 Hz, 1 H); 7.8 (m, 4 H).

Step 2

A mixture of the compound from step 1 (500 mg; 1.38 mmol), ethyl 2-(4′-fluorobiphenyl-4-yloxy)hex-5-enoate (589 mg; 1.79 mmol), dichlorobis-(tri-ortho-tolylphosphine) palladium II (108 mg; 0.14 mmol), tri-ortho-tolylphos-phine (42 mg; 0.14 mmol) and triethylamine (5 ml) in dimethylformamide (DMF) (4 ml) is heated for five hours in an oil bath at 110° C. The mixture is then poured into dilute hydrochloric acid and extracted with ethyl ether. The organic phase is dried over sodium sulfate and then concentrated. Flash chromatography (80/20 heptane/ethyl acetate) gives the expected product (0.49 g; 57%).

¹H NMR (300 MHz, chloroform-D), δ ppm: 1.08-1.37 (m, 5H); 1.99-2.61 (m, 2H); 3.70-3.84 (m, 3H); 4.11-4.33 (m, 2H); 4.48-4.81 (m, 1H); 4.48-4.81 (m, 1H); 5.03-5.09 (s, 2H); 6.13-6.35 (m, 1H); 6.63-7.02 (m, 6H); 7.03-7.16 (m, 2H); 7.33-7.68 (m, 8H).

### Example 2

### 2-(4′-Fluorobiphenyl-4-yloxy)-6-[5-methoxy-2-(4-trifluoromethyl-benzyloxy)phenyl]hex-5-enoic acid

A mixture of the compound of Example 1 (0.30 g; 0.49 mmol), ethanol (7 ml) and 1N potassium hydroxide (KOH) solution (2.5 ml) is heated at 60° C. for 4 hours. The mixture is then poured into water and extracted with ethyl ether. The aqueous phase is acidified with 1N hydrochloric acid and then extracted with dichloromethane. The organic phase is dried over sodium sulfate and then concentrated (195 mg). Flash chromatography (98/2 di-chloromethane/methanol) gives the expected product (120 mg; 42%).

LC/MS: ES−579.4

### Example 3

### Ethyl 6-[5-methoxy-2-(5-methyl-2-phenyloxazol-4-ylmethoxy)-phenyl]-2-(4-trifluoromethylphenoxy)hex-5-enoate

Step 1

A mixture of 2-bromo-4-methoxyphenol (1.0 g; 4.92 mmol) and 4-chloromethyl-5-methyl-2-phenyloxazole (1.125 g; 5.42 mmol) in acetone (50 ml) is refluxed for 11 hours. The mixture is filtered and the filtrate is concentrated to dryness. The residue is taken up in water and dichloromethane. The organic phase is washed with aqueous 1N sodium hydroxide (NaOH) and with water, dried and then concentrated (1 g; 54%).

¹H NMR (300 MHz, chloroform-D), δ ppm: 2.4 (s, 3 H); 3.8 (s, 3 H); 5.0 (s, 2 H); 6.8 (dd, J=9.0, 3.0 Hz, 1 H); 7.0 (d, J=9.0 Hz, 1 H); 7.1 (d, J=3 Hz, 1 H); 7.5 (m, 3 H); 8.0 (m, 2 H).

Step 2

A mixture of the compound from step 1 (288 mg; 0.77 mmol), ethyl 2-(4-trifluoromethylphenyl-4-yloxy)hex-5-enoate (541 mg; 1.79 mmol), dichlorobis(tri-ortho-tolylphosphine) palladium II (12.5 mg; 0.016 mmol), tri-ortho-tolylphosphine (19.5 mg; 0.06 mmol) and triethylamine (2.4 ml) in DMF (2 ml) is heated for 7 hours in an oil bath at 110° C. The mixture is then poured into dilute hydrochloric acid and extracted with ethyl ether. The organic phase is dried over sodium sulfate and then concentrated (0.8 g). Flash chromatography (80/20 heptane/ethyl acetate) gives the expected product (0.1 g; 21%).

¹H NMR (300 MHz, chloroform-D), δ ppm: 1.2 (t, J=7.1 Hz, 3 H); 2.1 (m, 2 H); 2.4 (m, 2 H); 2.3 (s, 3 H); 3.8 (s, 3 H); 4.2 (q, J=7.1 Hz, 2 H); 4.7 (dd, J=7.5, 5.1 Hz, 1 H); 4.9 (s, 2 H); 6.2 (m, 1 H); 6.7 (m, 2 H); 6.9 (m, 4 H); 7.5 (m, 5 H); 8.0 (m, 2 H).

LC/MS: ES+596.3

### Example 4

### 6-[5-Methoxy-2-(5-methyl-2-phenyloxazol-4-ylmethoxy)phenyl]-2-(4-trifluoromethylphenoxy)hex-5-enoic acid

A mixture of the compound of Example 3 (0.10 g; 0.17 mmol), methanol (8 ml) and 1N sodium hydroxide (NaOH) solution (0.835 ml) is heated at 60° C. for 2 hours. The mixture is then poured into water and extracted with ethyl ether. The aqueous phase is acidified with 1N hydrochloric acid and then extracted with ethyl ether. The organic phase is dried over sodium sulfate and then concentrated (50 mg). Flash chromatography (95/5 dichloromethane/methanol) gives the expected product (11 mg, 9%).

LCMS: ES+568.3

### Example 5

### Ethyl 6-(2-hydroxy-5-methoxyphenyl)-2-(2-methoxyphenoxy)-hex-5-enoate

Step 1

A mixture of 2-bromo-4-methoxyphenol (162 mg; 0.80 mmol), ethyl 2-(2-methoxylphenoxy)hex-5-enoate (296 mg; 0.89 mmol), dichlorobis(tri-ortho-tolylphosphine)palladium II (69 mg; 0.088 mmol), tri-ortho-tolylphosphine (21.4 mg; 0.070 mmol) and triethylamine (2.6 ml) in DMF (2 ml) is heated for 5 hours in an oil bath at 110° C. The mixture is then poured into dilute hydrochloric acid and extracted with ethyl ether. The organic phase is dried over sodium sulfate and then concentrated (0.59 g). Flash chromatography (70/30 heptane/ethyl acetate) gives the expected product (0.1 g, 32%).

¹H NMR (300 MHz, chloroform-D), δ ppm: 1.3 (t, J=7.2 Hz, 3 H); 2.1 (m, 2 H); 2.4 (m, 2 H); 3.9 (s, 3 H); 4.3 (q, J=7.1 Hz, 2 H); 4.7 (dd, J=8.0, 4.8 Hz, 1 H); 5.1 (m, 2 H); 5.9 (m, 1 H); 7.0 (m, 4 H)

LC/MS: ES−385.2/ES+409.1 (M+Na+)

Step 2

A mixture of the compound from step 1 (68 mg, 0.176 mmol), methanol (8 ml), water (4 ml) and 1N sodium hydroxide (NaOH) solution (0.8 ml) is heated at 60° C. for 5 hours. The mixture is then poured into water and extracted with ethyl ether. The aqueous phase is acidified with 1N hydrochloric acid and then extracted with dichloromethane. The organic phase is dried over sodium sulfate and then concentrated (15 mg, 85%).

LC/MS: ES−357.5

### Example 6:

### Ethyl 2-(2-methoxyphenoxy)-6-[5-methoxy-2-(4-trifluoromethyl-benzyloxy)phenyl]hex-5-enoate

A mixture of the compound from step 1 of Example 5 (300 mg; 0.78 mmol), 4-trifluoromethylbenzyl alcohol (117 μl; 1.1 eq.) and triphenyl-phosphine (215 mg; 0.82 mmol) in toluene (3.6 ml) is heated at 54° C. A solution of diisopropyl azodicarboxylate (DIAD) (158 μl; 1 eq.) in toluene (0.36 ml) is then added dropwise. The mixture is heated for 1.5 hours at 54° C. The medium is poured into water and extracted with ethyl ether. The organic phase is dried, concentrated (yellow oil, 0.8 g) and then purified by flash chromatography (80/20 heptane/ethyl acetate). An oil is obtained (0.28 g; 64%).

LC/MS: ES+545.3

### Example 7

### 2-(2-Methoxyphenoxy)-6-[5-methoxy-2-(4-trifluoromethylbenzyl-oxy)phenyl]hex-5-enoic acid

A mixture of the compound of Example 6 (0.28 g; 0.51 mmol), methanol (20 ml) and 1N sodium hydroxide solution (2.57 ml) is heated at 60° C. for 2 hours. The mixture is concentrated, taken up in water and extracted with ethyl ether. The aqueous phase is acidified with 1N hydrochloric acid and then extracted with ethyl ether. The organic phase is dried over sodium sulfate and then concentrated (99 mg). Flash chromatography (95/5 dichloromethane/methanol) gives the expected product (28 mg, 86%).

LC/MS: ES−515.3

### Example 8:

### Ethyl 2-(4-trifluoromethylphenoxy)-6-phenylhex-5-enoate

A mixture, under a nitrogen atmosphere, of ethyl 2-hydroxy-6-phenylhex-5-enoate (820 mg; 3.5 mmol), triphenylphosphine (1.38 g; 5.25 mmol; 1.5 eq.) and 4-trifluoromethylphenol (570 mg; 3.5 mmol; 1 eq.) in THF (35 ml) is cooled to 0° C. A solution of ethyl azodicarboxylate (DEAD) (900 mg; 5.25 mmol; 1.5 eq.) is added dropwise while maintaining this temperature. The mixture is then allowed to warm to room temperature and is stirred for 20 hours. The reaction medium is then concentrated to dryness under vacuum and taken up with stirring in 20 ml of ethyl ether. This mixture is filtered by suction and the solid formed is discarded. The filtrate is again concentrated to dryness under vacuum (yellow oil, 1.38 g) and then purified by flash chromatography (95/5 heptane/ethyl acetate). A colourless oil that crystallises slowly is obtained (780 mg, 60%).

¹H NMR (300 MHz, chloroform-D), δ ppm: 1.2 (t, J=7.2 Hz, 3 H); 2.1 (m, 2 H); 2.4 (m, 2 H); 4.1 (q, J=7.2 Hz, 2 H); 4.6 (dd, J=7.7, 4.7 Hz, 1 H); 6.1 (m, 1 H); 6.3 (m, 1 H); 6.9 (d, J=8.7 Hz, 2 H); 7.2 (m, 5 H); 7.5 (d, J=8.3 Hz, 2 H).

### Example 9

### 2-(4-Trifluoromethylphenoxy)-6-phenylhex-5-enoic acid

A mixture of the compound of Example 8 (700 mg; 1.85 mmol), ethanol (10 ml) and 85% potassium hydroxide (KOH) pellets (600 mg; 9.25 mmol; 5 eq.) is refluxed for 30 minutes. 5 ml of water are then added and refluxing is continued for 4 hours. The solution is then evaporated to dryness under vacuum and taken up in 15 ml of water. The cloudy solution obtained is filtered, acidified with 5N hydrochloric acid and extracted with ethyl ether. The organic phase is dried over sodium sulfate and then evaporated to dryness under vacuum. A yellowish-white solid is obtained (450 mg; 70%).

¹H NMR (300 MHz, chloroform-D), δ ppm: 2.07-2.31 (m, 2H); 2.34-2.62 (m, 2H); 4.76 (m, 1H); 6.18 (m, 1H); 6.38 (m, 1H); 6.96 (d, 2H, J=8.7 Hz); 7.10-7.37 (m, 5H); 7.55 (d, 2H, J=8.7 Hz).

Melting point: 107-108° C.

### Example 10

### Ethyl 2-[4-(5-chlorothien-2-yl)phenoxy]-6-phenylhex-5-enoate

To a mixture, under a nitrogen atmosphere, of ethyl 2-(4-bromo-phenoxy)-6-phenylhex-5-enoate (760 mg; 1.95 mmol), tetrakis-(triphenyl-phosphine)palladium (68 mg; 0.059 mmol; 0.03 eq.) and 2-chloro-5-thio-pheneboronic acid in 1,2-dimethoxyethane (monoglyme, 15 ml) is added dropwise a solution of sodium carbonate (450 mg; 4.3 mmol; 2.2 eq.) in water (3 ml). The mixture is refluxed for 4 hours and then stirred at room temperature overnight. The resulting mixture is poured into water, extracted with ethyl ether and washed with saturated aqueous sodium chloride solution. The organic phase is dried over sodium sulfate, evaporated to dryness under vacuum (brown oil, 730 mg) and then purified by flash chromatography (95/5 heptane/ethyl acetate). A yellow oil that crystallises is obtained (260 mg, 31%).

¹H NMR (300 MHz, chloroform-D), δ ppm: 1.3 (t, J=7.2 Hz, 3 H); 2.1 (m, 2 H); 2.5 (m, 2 H); 4.2 (q, J=7.2 Hz, 2 H); 4.7 (dd, J=7.9, 4.9 Hz, 1 H); 6.2 (m, 1 H); 6.4 (m, 1 H); 6.9 (m, 3 H); 6.9 (d, J=3.8 Hz, 1 H); 7.3 (m, 5 H); 7.4 (m, 2 H).

### Example 11

### 2-[4-(5-Chlorothien-2-yl)phenoxy]-6-phenylhex-5-enoic acid

A mixture of the compound of Example 10 (230 mg; 0.54 mmol), ethanol (10 ml) and 85% potassium hydroxide (KOH) pellets (180 mg; 2.7 mmol; 5 eq.) is refluxed for 30 minutes. 1.5 ml of water are then added and refluxing is continued for 4 hours. The solution is then evaporated to dryness and taken up in 20 ml of water. The suspension obtained is acidified with 5N hydrochloric acid, stirred for 30 minutes and extracted with ethyl acetate. The organic phase is dried over sodium sulfate and then evaporated to dryness under vacuum. A pale yellow solid is obtained (170 mg, 79%).

¹H NMR (300 MHz, chloroform-D), δ ppm: 2.16 (2H, m); 2.46 (2H, m); 4.73 (1H, m); 6.07-6.45 (2H, m); 6.74-6.98 (4H, m); 7.11-7.50 (7H, m).

Melting point: 158-160° C.

Compounds 12 to 59 were prepared according to protocols similar to those described for the preparation of the compounds of Examples 1 to 11 above.

The structures of compounds 12 to 59 are collated in Table 1 below,:

TABLE 1
Structures of compounds 12 to 59

| | |
|---|---|
| | (1) |
| 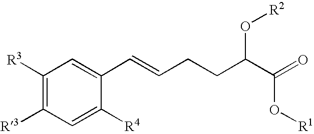 | |

| Ex. | | | | | |
|---|---|---|---|---|---|
| No. | R¹ | R² | R³ | R′³ | R⁴ |
| 12 | —CH₂—CH₃ | 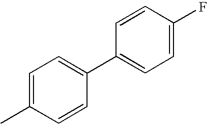 | —H | —H | —H |
| 13 | —H | 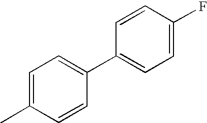 | —H | —H | —H |
| 14 | —H | 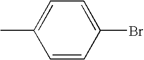 | —H | —H | —H |
| 15 | —H | 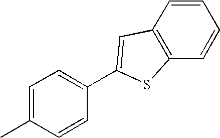 | —H | —H | —H |
| 16 | —H | 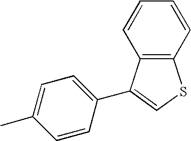 | —H | —H | —H |
| 17 | —CH₂—CH₃ | 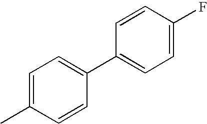 | 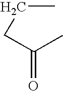 | | 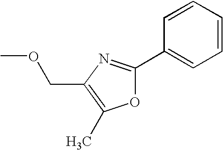 |
| 18 | —H | 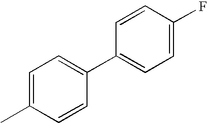 | 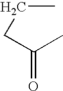 | | 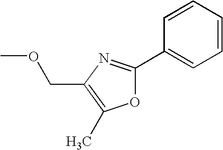 |
| 19 | —CH₂—CH₃ | 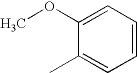 | 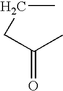 | | 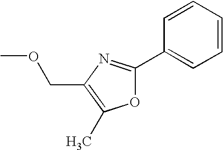 |
| 20 | —H | 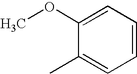 | 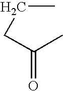 | | 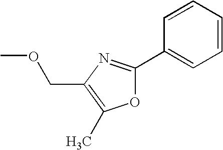 |
| 21 | —CH₂—CH₃ | 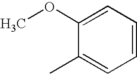 | —OCH₃ | —H | —OH |
| 22 | —CH₂—CH₃ | 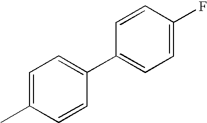 | —OCH₃ | —H | 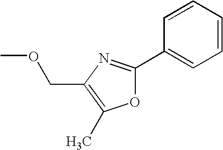 |
| 23 | —H | 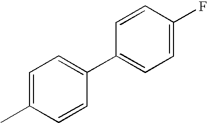 | —OCH₃ | —H | 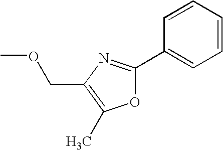 |
| 24 | —CH₂—CH₃ | —CH₂—CH₃ | —OCH₃ | —H | 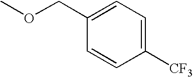 |
| 25 | —CH₂—CH₃ | 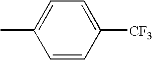 | —OCH₃ | —H | 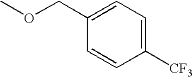 |
| 26 | —H | —CH₂—CH₃ | —OCH₃ | —H | 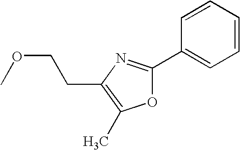 |
| 27 | —CH₂—CH₃ | —CH₂—CH₃ | —OCH₃ | —H | 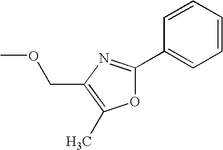 |
| 28 | —CH₂—CH₃ | 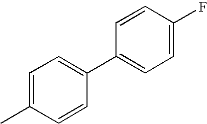 | —OCH₃ | —H | 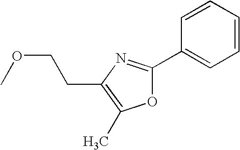 |
| 29 | —CH₂—CH₃ | 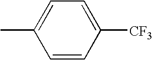 | —OCH₃ | —H | 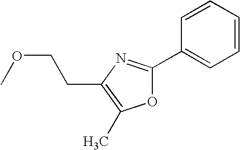 |
| 30 | —CH₂—CH₃ | 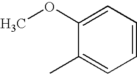 | —OCH₃ | —H | 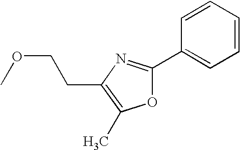 |
| 31 | —H | —CH₂—CH₃ | —OCH₃ | —H | 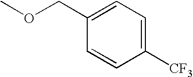 |
| 32 | —H | 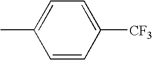 | —OCH₃ | —H | 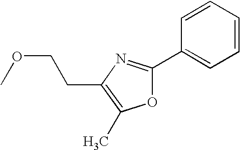 |
| 33 | —H | 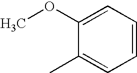 | —OCH₃ | —H | 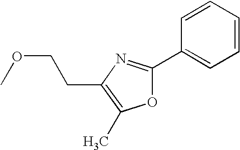 |
| 34 | —H | 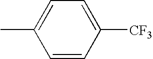 | —OCH₃ | —H | 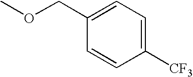 |
| 35 | —H | 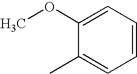 | —OCH₃ | —H | 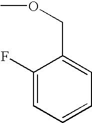 |
| 36 | —H | 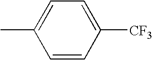 | —OCH₃ | —H | 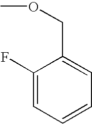 |
| 37 | —H | 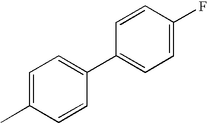 | —OCH₃ | —H | 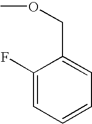 |
| 38 | —H | —CH₂—CH₃ | —OCH₃ | —H | 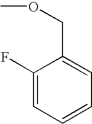 |
| 39 | —H | 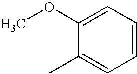 | —OCH₃ | —H | 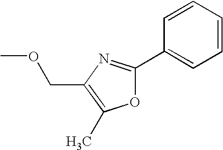 |
| 40 | —H | —CH₂—CH₃ | —OCH₃ | —H | 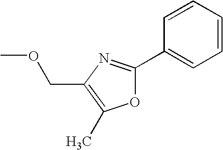 |
| 41 | —H | 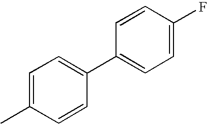 | 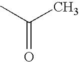 | —H | 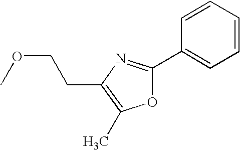 |
| 42 | —CH₂—CH₃ | 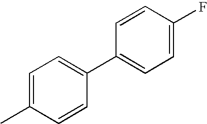 | —OCH₃ | —H | —OH |
| 43 | —H | 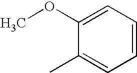 | 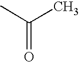 | —H | 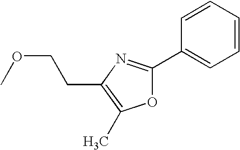 |
| 44 | —H | 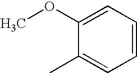 | —F | —H | 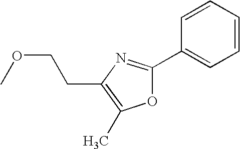 |
| 45 | —H | —CH₂—CH₃ | —F | —H | 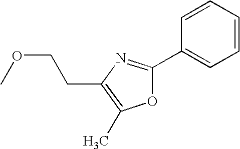 |
| 46 | —H | 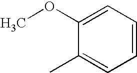 | —F | —H | 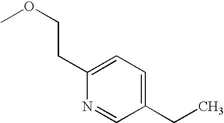 |
| 47 | —H | —CH₂—CH₃ | —F | —H | 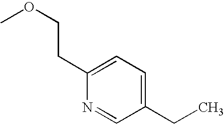 |
| 48 | —H | —CH₂—CH₃ | —CH₃ | —H | 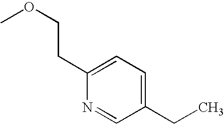 |
| 49 | —H | 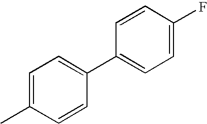 | —OCH₃ | —H | —OH |
| 50 | —CH₂—CH₃ | 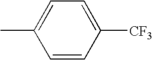 | —F | —H | 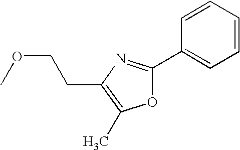 |
| 51 | —H | 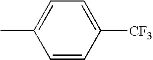 | —F | —H | 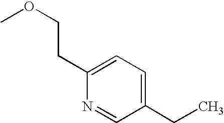 |
| 52 | —H | —CH₂—CH₃ | —OCH₃ | —H | —OH |
| 53 | —H | 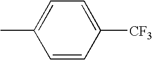 | —OCH₃ | —H | —OH |
| 54 | —H | 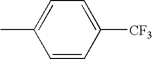 | —F | —H | —OH |
| 55 | —H | 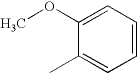 | —CH₃ | —H | —OH |
| 56 | —H | —CH₂—CH₃ | —CH₃ | —H | —OH |
| 57 | —H | 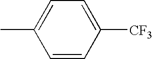 | —F | —H | 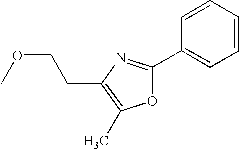 |
| 58 | —H | 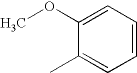 | —OCH₃ | —H | 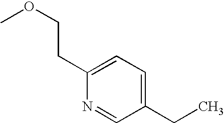 |
| 59 | —H | 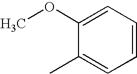 | —CH₃ | —H | 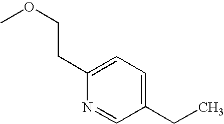 |

The results of the analyses of the synthesised products 6 to 31 are given in Table 2 below, in which table:

M represents the theoretical molar mass of the compound;

LC/MS indicates the result of the analysis by mass spectrometry coupled to liquid-phase chromatography;

m.p. represents the melting point in ° C.; and

NMR indicates the chemical shifts δ (in ppm) of the proton by magnetic resonance at 300 MHz.

TABLE 2

| Ex. No. | M | LC/MS | M.p. | NMR |
|---|---|---|---|---|
| 12 | 404.48 | | 80-82 | (CDCl₃): 1.26 (3H, t, J = 7.2 Hz); |
| | | | | 2.14 (2H, m); 2.46 (2H, m); |
| | | | | 4.22 (2H, q, J = 7.2 Hz); 4.69 (1H, m); |
| | | | | 6.08-6.52 (2H, m); 6.82-7.14 (4H, m); 7.15-7.36 |
| | | | | (5H, m); 7.36-7.57 (4H, m). |
| 13 | 376.42 | | 174-176 | (CDCl₃): 2.18 (2H, m); 2.47 (2H, |
| | | | | m); 4.76 (1H, m) 6.09-6.50 (2H, |
| | | | | m); 6.92-7.15 (4H, m); |
| | | | | 7.15-7.36 (5H, m); 7.39-7.56 (4H, m). |
| 14 | 361.23 | | 120 | (CDCl₃): 2.1 (m, 2H); 2.4 (m, 2H); |
| | | | | 4.6 (dd, J = 7.2, 5.3 Hz, 1H); |
| | | | | 6.1 (m, 1H); 6.3 (m, 1H); 6.7 (m, |
| | | | | 2H); 7.2 (m, 5H); 7.3 (m, 2H); |
| 15 | 414.52 | | 193-194 | (DMSO-d6): 1.94-2.17 (2H, m); |
| | | | | 2.27-2.46 (2H, m); 4.70-4.93 (1H, |
| | | | | m); 6.21-6.52 (2H, m); |
| | | | | 6.88-7.10 (2H, m); 7.10-7.49 (7H, m); |
| | | | | 7.59-8.07 (5H, m); 13.19 (1H, s élargi). |
| 16 | 414.52 | | 133-134 | (CDCl₃): 2.06-2.36 (2H, m); |
| | | | | 2.38-2.66 (2H, m); 4.67-4.90 (1H, m); |
| | | | | 6.11-6.32 (1H, m); 6.32-6.52 (1H, m); |
| | | | | 6.96-7.09 (2H, m); |
| | | | | 7.15-7.44 (8H, m); 7.44-7.58 (2H, m); |
| | | | | 7.79-8.03 (2H, m). |
| 17 | 645.72 | ES+ 646.4 | | |
| 18 | 617.67 | ES+ 618.3 | | |
| 19 | 581.66 | ES+ 582.3 | | |
| 20 | 553.61 | ES+ 554.3 | | |
| 21 | 386.44 | ES− 385.3 | | |
| 22 | 621.70 | ES+ 622.4 | | |
| 23 | 593.65 | ES+ 594.3 | | |
| 24 | 466.49 | ES+ 467.3 | | |
| 25 | 582.53 | ES+ 583.2 | | |
| 26 | 456.54 | ES+ 466.4 | | |
| 27 | 493.60 | ES+ 494.4 | | |
| 28 | 635.73 | ES+ 636.4 | | |
| 29 | 609.64 | ES+ 610.4 | | |
| 30 | 571.67 | ES+ 572.4 | | |
| 31 | 438.44 | ES− 437.4 | | |
| 32 | 581.58 | ES− 580.5 | | |
| 33 | 543.61 | ES− 542.5 | | |
| 34 | 554.48 | ES− 553.5 | | |
| 35 | 466.50 | ES− 465.5 | | |
| 36 | 504.47 | ES− 503.5 | | |
| 37 | 530.56 | ES− 529.5 | | |
| 38 | 388.43 | ES− 387.4 | | |
| 39 | 529.59 | ES+ 530.5 | | |
| 40 | 451.52 | ES+ 452.4 | | |
| 41 | 619.69 | ES− 618.4 | 80 | |
| 42 | 450.50 | ES+ 451.2 | | |
| 43 | 555.62 | ES+ 556.4 | | |
| 44 | 531.58 | ES+ 532.6 | | |
| 45 | 453.51 | ES+ 454.4 | | |
| 46 | 479.55 | ES+ 480.4 | | |
| 47 | 401.48 | ES+ 402.5 | | |
| 48 | 397.51 | ES+ 398.6 | | |
| 49 | 422.45 | ES− 421.5 | | |
| 50 | 597.60 | ES+ 598.5 | | |
| 51 | 517.52 | ES+ 518.4 | | |
| 52 | 280.32 | ES− 279.3 | | |
| 53 | 396.36 | ES− 395.4 | | |
| 54 | 384.32 | ES− 383.3 | | |
| 55 | 342.39 | ES− 341.4 | | |
| 56 | 264.32 | ES− 263.4 | | |
| 57 | 569.55 | ES+ 570.5 | | |
| 58 | 491.58 | ES+ 492.4 | | |
| 59 | 475.58 | ES+ 476.6 | | |

### Results

The measurement of the PPAR activation was performed according to a technique described by Lehmann et al. (J. Biol. Chem., 270, (1995), 12953-12956).

CV-1 cells (monkey kidney cells) are cotransfected with an expression vector for the chimeric protein PPARγ-Gal4 and with a “reporter” plasmid that allows expression of the luciferase gene placed under the control of a promoter comprising Gal4 response elements.

The cells are seeded in 96-well microplates and cotransfected using a commercial reagent with the reporter plasmid (pG5-tk-pGL3) and the expression vector for the chimeric protein (PPARγ-Gal4). After incubation for 4 hours, whole culture medium (comprising 10% foetal calf serum) is added to the wells. After 24 hours, the medium is removed and replaced with whole medium comprising the test products. The products are left in contact with the cells for 18 hours. The cells are then lysed and the luciferase activity is measured using a luminometer. A PPARγ activation factor can then be calculated by means of the activation of the expression of the reporter gene induced by the product (relative to the control cells that have received no product).

In the absence of the PPARγ ligand binding domain (vector expressing Gal4 alone), the luciferase activity measured in the presence of an agonist is zero.

The following transactivation result was obtained with a concentration of 10 μM on PPARγ.

| | | Activation factor of the |
|---|---|---|
| Ex. | Concentration | chimeric protein PPARγ-Gal4 |
| 44 | 10 μM | 5.8 |
| Without agonist | — | 1 |
| (Control) | | |

Example of Biological Activities of Partial Agonists

Transactivation Test

The transactivation test using the expression of a chimeric protein Gal-4-PPARγ makes it possible to determine also whether an agonist functions as a “full” agonist or as a “partial” agonist in this system.

An agonist is “partial” in this system if it induces a weaker response, i.e. it has lower efficacy, than rosiglitazone, which is a “full” agonist. In concrete terms, in our system, the transactivation obtained at the plateau with a partial agonist will be between 20% and 50% of the maximum response (efficacy) at the plateau of rosiglitazone.

| | Maximum stimulation of the | Concentration to reach the |
|---|---|---|
| | PPARγ chimeric protein | maximum stimulation of the PPARγ |
| Ex. | obtained with rosiglitazone | chimeric protein |
| 51 | 19% | 10 μM |

1. Compound of the formula (1): 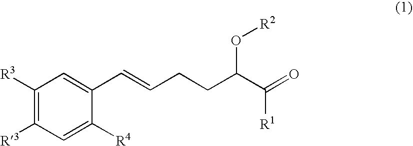
in which:
R¹ represents —O—R′¹ or —NR′¹R″¹, with R′¹ and R″¹, which may be identical or different, being chosen from a hydrogen atom, an alkyl radical, an alkenyl radical, an alkynyl radical, a cycloalkyl radical, an aryl radical and a heteroaryl radical;
R² is chosen from:
an alkyl, alkenyl or alkynyl radical;
an aryl radical, optionally substituted and/or optionally fused to a monocyclic or polycyclic, saturated or unsaturated 5- to 8-membered nucleus optionally containing one or more hetero atoms chosen from O, N and S, the said nucleus itself being optionally substituted, and
a saturated, unsaturated or aromatic, optionally substituted 5- to 8-membered monocyclic heterocyclic radical containing one or more hetero atoms chosen from O, N and S;
R³ is chosen from a hydrogen atom; a halogen atom chosen from chlorine, fluorine, bromine and iodine, preferably fluorine; an alkyl radical; an alkoxy radical; and an alkylcarbonyl radical;
or alternatively R³ forms with R′³, and with the carbon atoms bearing the substituents R³ and R′³, an optionally substituted, 5- or 6-membered carbocyclic radical;
R′³ represents a hydrogen atom or alternatively forms with R³ and with the carbon atoms bearing the substituents R³ and R′³, an optionally substituted 5- or 6-membered carbocyclic radical; and
R⁴ is chosen from a hydrogen atom, the hydroxyl radical and a radical —O-A-R⁵, in which
A represents a linear or branched alkylene chain containing from 1 to 6 carbon atoms; and
R⁵ is chosen from an optionally substituted aryl radical and an optionally substituted heterocyclic radical;
the possible optical isomers, oxide forms and solvates thereof, and also pharmaceutically acceptable addition salts thereof with acids or bases.

2. Compound according to claim 1, having one or more of the following characteristics, taken separately or as a combination of one, several or all of them:
R¹ represents —O—R′¹, R′¹ being chosen from a hydrogen atom, an alkyl radical, an alkenyl radical, an alkynyl radical, a cycloalkyl radical, an aryl radical and a heteroaryl radical;
R² represents an alkyl radical or an optionally substituted aryl radical;
R³ is chosen from a hydrogen atom, a halogen atom chosen from chlorine, fluorine, bromine and iodine, preferably fluorine; an alkyl radical; an alkoxy radical; and an alkylcarbonyl radical;
or alternatively R³ forms with R′³ and with the carbon atoms bearing the substituents R³ and R′³, an optionally substituted 5- or 6-membered carbocyclic radical;
R′³ represents a hydrogen atom or forms with R³, and with the carbon atoms bearing the substituents R³ and R′³, an optionally substituted 5- or 6-membered carbocyclic radical; and
R⁴ is chosen from a hydrogen atom, a hydroxyl radical and a radical —O-A-R⁵, in which
A represents a linear or branched alkylene chain containing from 1 to 6 carbon atoms; and
R⁵ is chosen from an optionally substituted aryl radical and an optionally substituted heterocyclic radical;
the possible optical isomers, oxide forms and solvates thereof, and also the pharmaceutically acceptable addition salts thereof with acids or bases.

3. Compound according to claim 1, having one or more of the following characteristics, taken separately or as a combination of one, several or all of them:
R¹ represents —O—R′¹, R′¹ being chosen from a hydrogen atom, an alkyl radical, a cycloalkyl radical, an aryl radical and a heteroaryl radical;
R² represents a C₁-C₆ alkyl radical or an optionally substituted phenyl radical;
R³ is chosen from a hydrogen atom, a halogen atom chosen from chlorine, fluorine, bromine and iodine, preferably fluorine; a C₁-C₆ alkyl radical; a C₁-C₆ alkoxy radical; and a C₁-C₆ alkylcarbonyl radical;
or alternatively R³ forms with R′³ and with the carbon atoms bearing the substituents R³ and R′³, an optionally substituted 5-membered carbocyclic radical;
R′³ represents a hydrogen atom or forms with R³, and with the carbon atoms bearing the substituents R³ and R′³, an optionally substituted 5-membered carbocyclic radical; and
R⁴ is chosen from a hydrogen atom, a hydroxyl radical and a radical —O-A-R⁵, in which
A represents a linear alkylene chain corresponding to the formula —(CH₂)ₖ— in which k represents an integer chosen from 1, 2, 3, 4, 5 and 6; and
R⁵ is chosen from an optionally substituted phenyl radical and an optionally substituted heterocyclic radical;
the possible optical isomers, oxide forms and solvates thereof, and also the pharmaceutically acceptable addition salts thereof with acids or bases.

4. Compound according to claim 1, having one or more of the following characteristics, taken separately or as a combination of one, several or all of them:
R¹ represents —O—R′¹, R′¹ being chosen from a hydrogen atom and a methyl, ethyl, propyl or isopropyl radical;
R² represents a methyl, ethyl, propyl or isopropyl radical, or a substituted phenyl radical;
R³ is chosen from a hydrogen atom, a fluorine atom, a methyl, ethyl, propyl or isopropyl radical, a methoxy, ethoxy, propoxy or isopropoxy radical, and a methylcarbonyl, ethylcarbonyl or propylcarbonyl radical;
or alternatively R³ forms with R′³, and with the carbon atoms bearing the substituents R³ and R′³, a substituted 5-membered carbocyclic radical;
R′³ represents a hydrogen atom or forms with R³, and with the carbon atoms bearing the substituents R³ and R′³, a substituted 5-membered carbocyclic radical; and
R⁴ is chosen from a hydrogen atom, a hydroxyl radical and a radical —O-A-R⁵, in which
A represents a linear alkylene chain corresponding to the formula —(CH₂)ₖ— in which k represents an integer chosen from 1, 2 and 3; and
R⁵ is chosen from a substituted phenyl radical and a substituted 5- or 6-membered heterocyclic radical;
the possible optical isomers, oxide forms and solvates thereof, and also the pharmaceutically acceptable addition salts thereof with acids or bases.

5. Compound according to claim 1, having one or more of the following characteristics, taken separately or as a combination of one, several or all of them:
R¹ represents —O—R′¹, R′¹ being chosen from a hydrogen atom, a methyl radical and an ethyl radical;
R² represents a methyl or ethyl radical, or a substituted phenyl radical;
R³ is chosen from a hydrogen atom, a fluorine atom, a methyl or ethyl radical, a methoxy or ethoxy radical, and a methylcarbonyl or ethylcarbonyl radical;
or alternatively R³ forms with R′³, and with the carbon atoms bearing the substituents R³ and R′³, a saturated 5-membered carbocyclic radical substituted by an oxo group;
R′³ represents a hydrogen atom or forms with R³, and with the carbon atoms bearing the substituents R³ and R′³, a saturated 5-membered carbocyclic radical substituted by an oxo group; and
R⁴ is chosen from a hydrogen atom, a hydroxyl radical and a radical —O-A-R⁵, in which
A represents an alkylene chain corresponding to the formula —(CH₂)ₖ— in which k represents an integer chosen from 1 and 2; and
R⁵ is chosen from a substituted phenyl radical and a substituted 5- or 6-membered heterocyclic radical comprising not more than three hetero atoms and preferably not more than two hetero atoms, chosen from O, N and S;
the possible optical isomers, oxide forms and solvates thereof, and the pharmaceutically acceptable addition salts thereof with acids or bases.

6. Compound according to claim 1, characterised in that the substituents on the aryl and heterocyclic radicals are chosen from methyl, ethyl, methoxy, phenyl, fluorine and trifluoromethyl,
the possible optical isomers, oxide forms and solvates thereof, and also pharmaceutically acceptable addition salts thereof with acids or bases.

7. Compound according to claim 1, characterised in that the heterocyclic radicals are chosen from thienyl, benzothiophenyl, pyridyl and oxazolyl radicals,
the possible optical isomers, oxide forms and solvates thereof, and also pharmaceutically acceptable addition salts thereof with acids or bases.

8. Compound according to claim 1, chosen from:
6-[5-methoxy-2-(5-methyl-2-phenyloxazol-4-ylmethoxy)phenyl]-2-(4-trifluoromethylphenoxy)hex-5-enoic acid;
ethyl 2-(4′-fluorobiphenyl-4-yloxy)-6-[5-methoxy-2-(5-methyl-2-phenyloxazol-4-ylmethoxy)phenyl]hex-5-enoate;
6-{5-methoxy-2-[2-(5-methyl-2-phenyloxazol-4-yl)ethoxy]phenyl}-2-(4-trifluoromethylphenoxy)hex-5-enoic acid;
2-(4′-fluorobiphenyl-4-yloxy)-6-[5-methoxy-2-(4-trifluoromethylbenzyloxy)-phenyl]hex-5-enoic acid;
6-[2-(2-fluorobenzyloxy)-5-methoxyphenyl]-2-(4′-fluorobiphenyl-4-yloxy)hex-5-enoic acid;
2-ethoxy-6-{5-fluoro-2-[2-(5-methyl-2-phenyloxazol-4-yl)ethoxy]phenyl}hex-5-enoic acid;
6-{2-[2-(5-ethylpyridin-2-yl)ethoxy]-5-methoxyphenyl}-2-(2-methoxy-phenoxy)hex-5-enoic acid;
6-{2-[2-(5-ethylpyridin-2-yl)ethoxy]-5-fluorophenyl}-2-(2-methoxyphenoxy)hex-5-enoic acid;
ethyl 6-{5-fluoro-2-[2-(5-methyl-2-phenyloxazol-4-yl)ethoxy]phenyl}-2-(4-trifluoromethylphenoxy)hex-5-enoate;
6-{5-fluoro-2-[2-(5-methyl-2-phenyloxazol-4-yl)ethoxy]phenyl}-2-(4-trifluoro-methylphenoxy)hex-5-enoic acid;
6-{5-Methoxy-2-[2-(5-methyl-2-phenyloxazol-4-yl)ethoxy]phenyl}-2-(2-methoxyphenoxy)hex-5-enoic acid;
6-[2-(2-fluorobenzyloxy)-5-methoxyphenyl]-2-(2-methoxyphenoxy)hex-5-enoic acid;
ethyl 6-[5-methoxy-2-(5-methyl-2-phenyloxazol-4-ylmethoxy)phenyl]-2-(4-trifluoromethylphenoxy)hex-5-enoate;
2-ethoxy-6-{2-[2-(5-ethylpyridin-2-yl)ethoxy]-5-fluorophenyl}hex-5-enoic acid;
2-(2-methoxyphenoxy)-6-[5-methoxy-2-(4-trifluoromethylbenzyloxy)phenyl]hex-5-enoic acid;
6-{5-fluoro-2-[2-(5-methyl-2-phenyloxazol-4-yl)ethoxy]phenyl}-2-(2-methoxy-phenoxy)hex-5-enoic acid;
6-{2-[2-(5-ethylpyridin-2-yl)ethoxy]-5-methylphenyl}-2-(2-methoxyphenoxy)hex-5-enoic acid;
6-{5-acetyl-2-[2-(5-methyl-2-phenyloxazol-4-yl)ethoxy]phenyl}-2-(4′ -fluoro-biphenyl-4-yloxy)hex-5-enoic acid;
6-{5-acetyl-2-[2-(5-methyl-2-phenyl-oxazol-4-yl)ethoxy]phenyl}-2-(2-meth-oxyphenoxy)hex-5-enoic acid;
6-{2-[2-(5-ethylpyridin-2-yl)ethoxy]-5-fluorophenyl}-2-(4-trifluoromethyl-phenoxy)hex-5-enoic acid;
6-[5-methoxy-2-(5-methyl-2-phenyloxazol-4-ylmethoxy)phenyl]-2-(2-meth-oxyphenoxy)hex-5-enoic acid;
ethyl 2-ethoxy-6-[5-methoxy-2-(4-trifluoromethylbenzyloxy)phenyl]hex-5-enoate;
2-ethoxy-6-[5-methoxy-2-(4-trifluoromethylbenzyloxy)phenyl]hex-5-enoic acid;
2-ethoxy-6-[2-(2-fluorobenzyloxy)-5-methoxyphenyl]hex-5-enoic acid;
2-(4′-fluorobiphenyl-4-yloxy)-6-[5-methoxy-2-(5-methyl-2-phenyloxazol-4-ylmethoxy)phenyl]hex-5-enoic acid;
2-ethoxy-6-{5-methoxy-2-[2-(5-methyl-2-phenyloxazol-4-yl)ethoxy]phenyl}hex-5-enoic acid;
2-ethoxy-6-[5-methoxy-2-(5-methyl-2-phenyloxazol-4-ylmethoxy)phenyl]hex-5-enoic acid;
2-ethoxy-6-{2-[2-(5-ethylpyridin-2-yl)ethoxy]-5-methylphenyl}-hex-5-enoic acid;
ethyl 2-ethoxy-6-{5-methoxy-2-[2-(5-methyl-2-phenyloxazol-4-yl)ethoxy]-phenyl}hex-5-enoate;
2-[4-(5-chlorothiophen-2-yl)phenoxy]-6-phenylhex-5-enoic acid;
ethyl 2-(4′-fluorobiphenyl-4-yloxy)-6-[6-(5-methyl-2-phenyloxazol-4-ylmethoxy)-1-oxoindan-5-yl]hex-5-enoate;
2-(4′-fluorobiphenyl-4-yloxy)-6-[6-(5-methyl-2-phenyloxazol-4-ylmethoxy)-1-oxoindan-5-yl]hex-5-enoic acid;
ethyl 2-(2-methoxyphenoxy)-6-[6-(5-methyl-2-phenyloxazol-4-ylmethoxy)-1-oxoindan-5-yl]hex-5-enoate; and
2-(2-methoxyphenoxy)-6-[6-(5-methyl-2-phenyloxazol-4-ylmethoxy)-1-oxoindan-5-yl]hex-5-enoic acid,
and from the possible optical isomers, oxide forms and solvates, and also the pharmaceutically acceptable addition salts with acids or bases, of these compounds.

9. Process for the preparation of a compound according to claim 1 by coupling a compound of the formula (2) with a hex-5-enoic acid derivative of the formula (3): 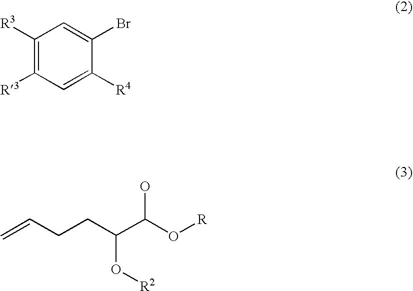
in which formulae R², R³, R′³ and R⁴ are as defined above for the formula (1) and R represents a protecting group for the acid function,
the said coupling being performed by catalysis in the presence of a phosphine and in the presence of a weak base, in polar medium, for a time ranging from one to several hours, so as to obtain the compound of the formula (1_{R}): 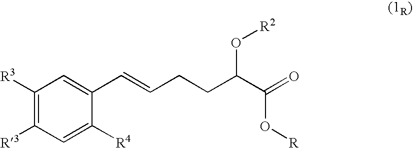
in which R², R³, R′³ and R⁴ are as defined for the formula (1) and R represents the protecting group of the acid function defined above,
which compound of the formula (1_{R}) is converted into the corresponding acid of the formula (1_{OH}): 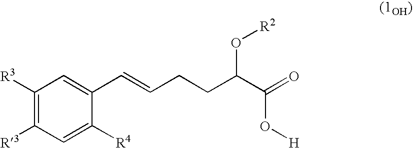
which is a special case of the compounds of the formula (1) in which R¹ represents a hydroxyl radical,
and the acid is optionally esterified, or converted into the corresponding amide, also according to standard techniques, to give the compound of the formula (1) with R¹ other than a hydroxyl radical.

10. Process for the preparation of a compound according to claim 1 by coupling a compound of the formula (3) with a compound of the formula (2_{OH}): 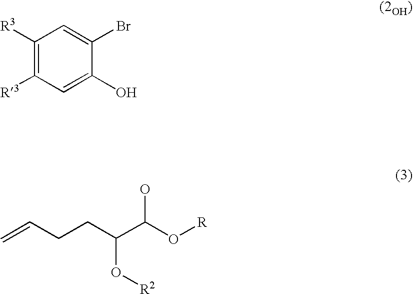
in which formulae R², R³ and R′³ are as defined above for the formula (1) and R represents a protecting group for the acid function,
the said coupling being performed by catalysis in the presence of a phosphine and in the presence of a weak base, in polar medium, for a time ranging from one to several hours, so as to obtain a compound of the formula (1′_{OH}): 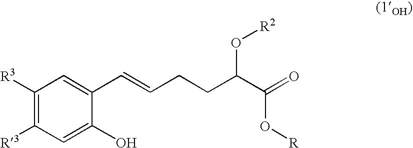
in which R, R², R³ and R′³ are as defined above,
the hydroxyl function borne by the phenyl nucleus being optionally converted into the various substituents defined for R⁴ in the formula (1), the group R then being optionally removed to give the acid function, and consequently the compounds of the formula (1_{OH}): 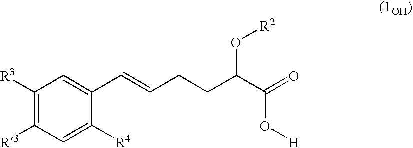
which is a special case of the compounds of the formula (1) in which R¹ represents a hydroxyl radical,
and the acid is optionally esterified, or converted into the corresponding amide, also according to standard techniques, to give the compound of the formula (1) with R¹ other than a hydroxyl radical.

11. Pharmaceutical composition comprising a pharmaceutically effective amount of at least one compound of the formula (1) or obtained via a process according to claim 9, in combination with one or more pharmaceutically acceptable vehicles.

12. Use of a compound of the formula (1) or obtained via a process according to claim 9, for the preparation of a medicament for the prevention or treatment of dyslipidaemia, atherosclerosis and diabetes.

13. A method of treating dyslipidaemia, atherosclerosis and diabetes comprising administering a compound of claim 1.
